(19) United States
(12) Patent Application Publication
(10) Pub. No.: US 2017/0261200 A1
(43) Pub. Date: Sep. 14, 2017

(54) DESKTOP VAPORIZER

(71) Applicant: David Stultz, Odessa, TX (US)

(72) Inventor: David Stultz, Odessa, TX (US)

(21) Appl. No.: 15/210,608

(22) Filed: Jul. 14, 2016

Related U.S. Application Data
(63) Continuation-in-part of application No. 29/557,830, filed on Mar. 11, 2016.

(51) Int. Cl. F22B 3/02; A24F 47/00; A61M 15/00; A61M 16/00; A61M 16/10; A61M 16/20

(52) U.S. Cl. CPC F22B 3/02; A61M 16/109; A61M 16/20; A61M 15/0086; A61M 16/0078; A24F 47/004; A61M 2205/3368; A61M 2205/59; A61M 2205/502

(57) ABSTRACT

A system and method for the production and capturing of clean vapors from the vaporizing of a plant substance is described. The system includes a heating system configured to generate heat within a cooking unit. A temperature control system is configured to monitor the level of heat generated by the heating system. Both systems are regulated by a control unit. The control unit is configured to provide an interface for a user to regulate the performance of the temperature control system and the heating system. The cooking unit defines a fully enclosed volume to contain the heat generated. A valve body is in communication with the cooking unit and configured to regulate the release of gases within the cooking unit as it passes into a bag member. The bag member is used to capture released vapor generated by vaporizing the plant substance.

### CLAIM OF PRIORITY

This application claims the benefit of U.S. Design application Ser. No. 29/557,830, filed 11 Mar. 2016. The information contained therein is hereby incorporated by reference.

### BACKGROUND

1. Field of the Invention

The present application relates to a compact cooking unit, and more particularly to a device used to vaporize a substance and capture the vapor therein.

2. Description of Related Art

Various plant substances are commonly used to provide recreational and medicinal health benefits. One typical way of administering the plant substance is through inhalation after the substance has been heated. This process involves heating the substance until it burns and in turn releases a vapor remnant. The vapor remnant is then inhaled by the user for his/her elected purpose. This is process is performed daily through cigarettes, where tobacco is burned and then inhaled. A disadvantage of this process is the harmful byproducts remaining in the vapor through burning the substance. This same disadvantage is likewise seen with other substances that are burned or inhaled in vapor form. Marijuana us another typical plant substance often inhaled by a user. There are many ways in which these plant substances are processed in order to create the vapor for inhalation.

As stated above, a common disadvantage of many systems is that the plant substances are actually burned, thereby releasing other contaminants. Additionally, systems tend to require a user to inhale the vapor during the burning or shortly thereafter wherein the vapor emitted is still hot and can burn the user. Furthermore, often there is a lack of control of the precise temperature at which the plant substance is exposed.

Although great strides have been made in the forgoing systems, considerable shortcomings remain. A new system is needed that provides control to the user with respect to heating the plant substance in order to avoid burning. Additionally, increased control of the heating systems are needed as well as a method of capture of the vapor to allow for it to cool prior to inhalation.

### SUMMARY OF THE INVENTION

The present application discloses a system that is configured to be a compact desktop unit designed for the purpose of accepting a substance for heating and for the selected capture and release of remnant vapor released thereby. The system is configured to include an outer housing with an internal cooking unit. The cooking unit is operated through a temperature control system and a heating system. A user is able to control the functions of the temperature control system and the heating system through a control unit interface.

Operation of this device is configured to permit a user the ability to insert a desired amount of plant substance into the cooking unit and selectively control the duration and temperature at which the substances are heated. Vapor released through the heating process is passed through a valve bod and captured within an external bag member. A user is able to selectively remove the bad member for inhalation of the vapor.

The invention may take many embodiments but features the ability to more accurately control and regulate the temperature and heating systems to avoid escalating the level of heat to a level wherein the substance burns. Additionally, the vapor is externally captured outside of the cooking unit/vaporizer unit and is removable through a bag member wherein the user is able to wait to inhale after the temperature of the vapor has lowered. The external bag member also permits the user to more quickly vaporize substances by replacing the bag member with a replacement bag member for filling with vapor while the first bag member is consumed. In these ways, this system overcomes the disadvantages inherent in existing methods and system described above.

The more important features of the system have thus been outlined in order that the more detailed description that follows may be better understood and to ensure that the present contribution to the art is appreciated. Additional features of the system will be described hereinafter and will form the subject matter of the claims that follow.

Many objects of the present system will appear from the following description and appended claims, reference being made to the accompanying drawings forming a part of this specification wherein like reference characters designate corresponding parts in the several views.

Before explaining at least one embodiment of the system in detail, it is to be understood that the system is not limited in its application to the details of construction and the arrangements of the components set forth in the following description or illustrated in the drawings. The system is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present system. It is important, therefore, that the claims be regarded as including such equivalent constructions insofar as they do not depart from the spirit and scope of the present system.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a schematic of a desktop vaporizer system according to an embodiment of the present application.

FIG. 2 is a perspective view of an exemplary desktop vaporizer as seen in the schematic of FIG. 1.

FIG. 3 is a front view of the desktop vaporizer of FIG. 2.

FIG. 4 is a side view of the desktop vaporizer of FIG. 3.

FIG. 5 is a top view of the desktop vaporizer of FIG. 3.

FIG. 6 is a front view of the desktop vaporizer of FIG. 3 with a bag member filled with a vapor.

While the device and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the system and devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system and method in accordance with the present application overcomes one or more of the above-discussed problems commonly associated with traditional plant substance heating systems as described above. In particular, the device is configured to allow for the external capturing of vapors from a cooking unit/vaporizing unit to allow a user the ability to cool the vapors prior to inhaling the vapors. Additionally, the system includes a control unit configured to regulate the level of heat within the cooking unit. A valve body is used to regulate the transition of vapors from the cooking unit into the bag member for collection. The system permits a user particular control of the heating temperature so as to avoid burning of the plant substance. These and other unique features of the device are discussed below and illustrated in the accompanying drawings.

The system and method will be understood, both as to its structure and operation, from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system and method of the present application is illustrated in the associated drawings. The system includes a heating system configured to generate heat within a cooking unit. It also includes a temperature control system configured to monitor the level of heat generated by the heating system. The heating system and temperature control system is regulated by a control unit. The control unit is configured to provide an interface for a user to regulate the performance of the temperature control system and the heating system. The cooking unit defines a fully enclosed volume to capture the heat generated by the heating system. Furthermore, the system includes a valve body in communication with the cooking unit being configured to regulate the release of gases within the cooking unit. A bag member is also includes and is used to selectively couple to the valve body and capture released vapor generated by vaporizing the plant substance. Additional features and functions of the system are illustrated and discussed below.

Referring now to the drawings wherein like reference characters identify corresponding or similar elements in form and function throughout the several views.

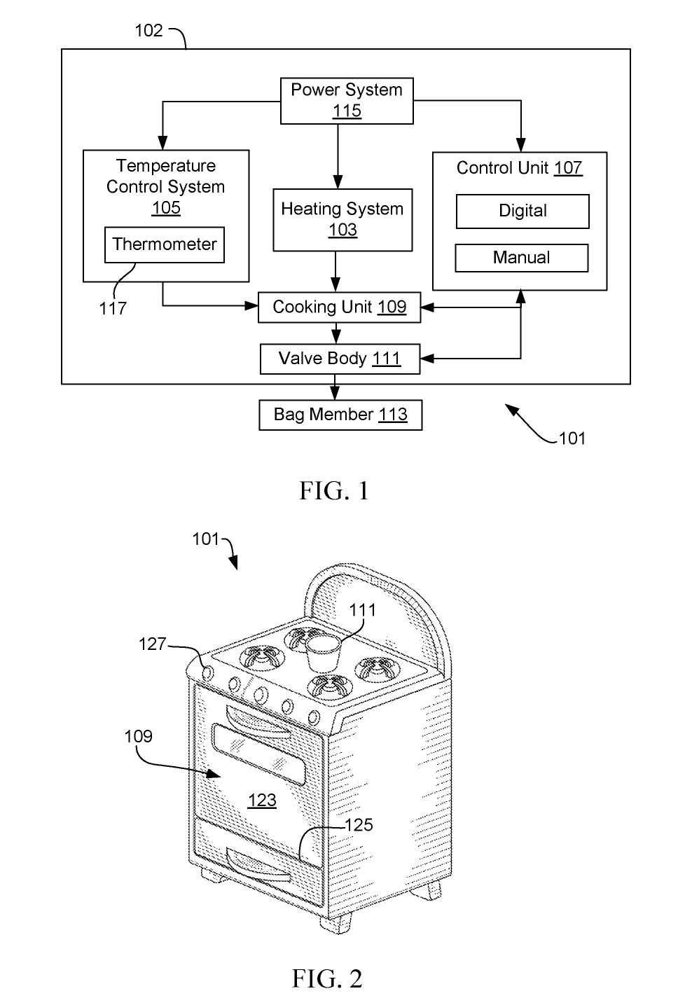

FIG. 1 illustrates a schematic of a desktop vaporizer system according to the present application. System 101 includes at least a bag member 113 and a vaporizer unit 102 having a heating system 103, a temperature control system 105, a control unit 107, a cooking unit 109, and a valve body 111.

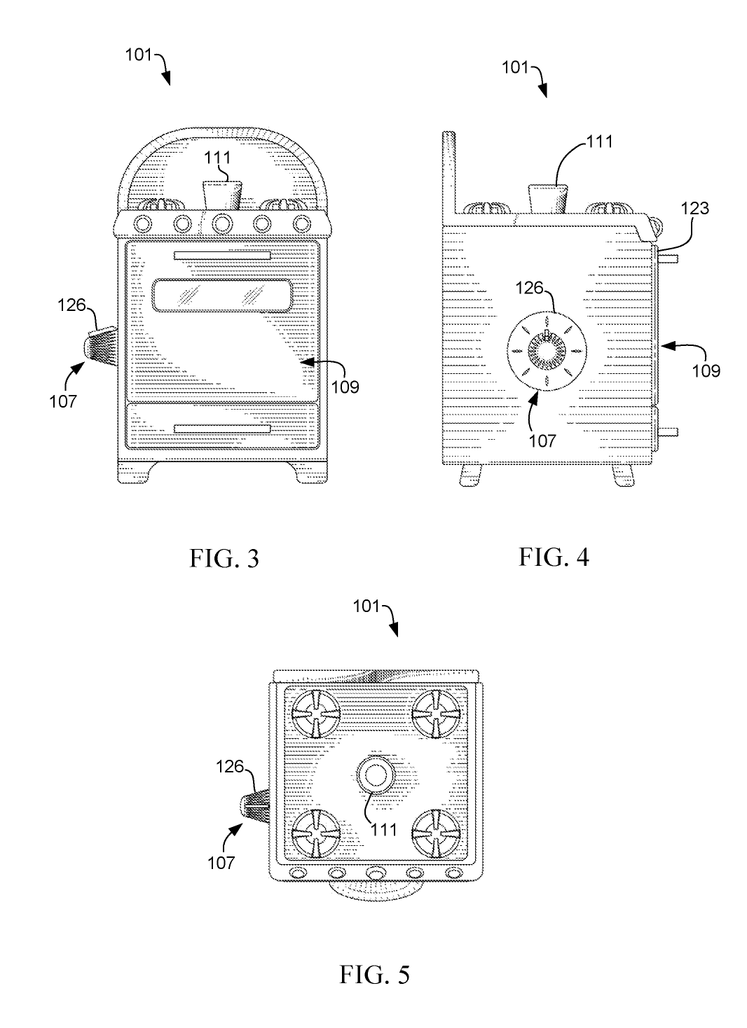

System 101 is designed to be compact and portable so as to be storable on a desk or counter. System 101 includes a power system 115 that is configured to provide sufficient power to each system and unit within system 101 in order to function as described. Power system 101 may be a battery that is replaceable and/or rechargeable, or may be adapted for communication with the power grid through a wall outlet. It is understood that system 101 is only schematically illustrated in FIG. 1. FIGS. 2-6 are used to show an exemplary embodiment of system 101.

Vaporizer unit 102 is configured to selectively heat and vaporize a plant substance within a cooking unit 109. A plant substance can refer to any plant, such as tobacco, marijuana, an herb or other for example. The cooking unit includes a plurality of walls that define an internal volume. Unit 109 is configured to capture heat generated from heating system 103. Heating system 103 is in communication with cooking unit 109 and is configured to provide heat within the internal volume. Temperature control system 105 is also configured to communicate with cooking unit 109 and heating system 103. System 105 is configured to monitor the level of heat generated by heating system 103. This is ideally done through a thermometer 117 used similarly to that of a conventional oven. Other methods of determining the temperature within cooking unit 109 is realized and are operable within system 101. Heat within unit 109 is configured to vaporize the plant substance. The level of heat provided is ideally less than the temperatures required to burn the plant substance. By eliminating the burning of the plant substance, toxins are avoided from being produced and mixed within the remnant vapor.

Control unit 107 is configured to provide an interface for a user to regulate the performance of systems 103 and 105. A user is able to operate unit 107 so as to select a temperature level and even the duration of heating. This may be done through manual controls or through digital interfaces. Temperature control system 105 is configured to record temperature data and transmit that data to control unit 107. Control unit 107 receives the temperature data and automatically regulates system 103 in order to maintain the desired temperature level of the user.

As the plant substance is vaporized within cooking unit 109, vapors are released. Valve body 111 is in communication with the internal volume of cooking unit 109 and is configured to selectively release the vapors/gases produced as a result of the vaporization. Valve body 111 may be interchanged to allow for different functional capabilities, such as flow rate, contour, attachment methods to bag member 113 and so forth. Vapors passing through valve body 111 are communicated to bag member 113.

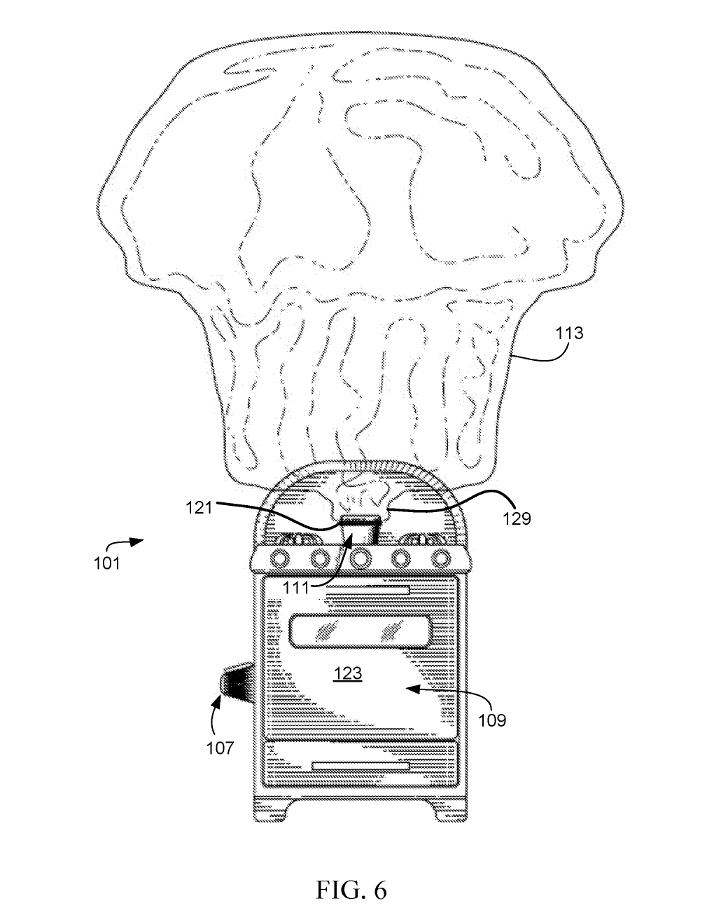

Bag member 113 is configured to store vapors for the eventual use of the operator/user. Bag member 113 is configured to releasably couple to an outer portion of valve body 111. Valve body 111 may be configured to grasp or secure bag member 113 or a separate attachment member may be used, such as a band 121 (see FIG. 6). At any point in the filling process, bag member 113 may be removed. An advantage of using bag member 113 to externally capture vapors is the ability to allow those vapors to cool prior to their eventual inhalation. This avoids issues where discomfort arise with hot vapors. Additionally, bag member 113 grants the user the ability to merely pinch or close off the singular opening and transport the vapors away from vaporizer unit 102. Other bag members may be attached in its place.

As seen in FIGS. 2-6 in the drawings, system 101 is depicted in various different views. System 101 is depicted as a small oven unit. Other shapes and styles are realized and considered capable within the scope of the present application. From the assorted views, cooking unit 109 is visible as being the volume of area behind door 123. Door 123 is configured to hingedly open along lower edge 125 so as to function like a conventional oven door.

Control unit 107 is illustrated in a manual mode. The body of unit 107 is seen as a knob 126 that is configured to rotate about an axis. Rotation of the knob in selected angular directions operates heating system 103. System 103 may be turned on or off. Additionally, the amount of rotation may elect the desired temperature to be obtained within cooking unit 109. A user may merely select the particular temperature level. Additionally, knob 126 may be configured to accept translational movement along its axis of rotation in order to perform a particular function. Other visible features seen on system 101 may be either ornamental in nature or serve some particular function. For example, knobs 127may be used to operate as a push button to set heating times, heat cycles, power level, and so forth. The use of power system 115 allows sufficient energy to digitize any portion of control unit 107, including knobs 127 and knob 126.

Bag member 113 is configured to selective attach to valve body 111. Member 113 may be designed to form into any particular shape. The shape depicted is not meant to be limiting. In FIG. 6, member 113 is shaped as a muffin. Other shapes are possible, such as characters, objects, food, and so forth. Member 113 ideally has a single entry/exit port 129 configured to communicate with valve body 111.

In operation, a user opens door 123 along edge 125 and inserts the desired plant substance. The user then uses control unit 107 to set the desired temperature level. Other controllable features may be selecting the duration of heating and the power level. The user vaporizes the plant substance in cooking unit 109 and captures the resulting vapors in member 113. The user has now externally captured the vapors away from unit 109 and can remove bag member 113 as desired for inhalation of the vapors. It is understood that bag member 113 is typically a flexible member that expands as it is filled. Other rigid forms may be used as well. Member 113 does not have to be flexible. For example, bag member 113 may be rigid wherein portion 129 is configured to sealingly engage valve body 111 thereby resulting in no use or need for band 121 (an attachment member). Any portion of bag member 113 may be flexible and/or rigid to ease capturing vapors, coupling to valve body 111, and consumption by the user.

The current application has many advantages over the prior art including at least the following: (1) the precise temperature controlled vaporizing of the plant substance in order to eliminate the production of toxins from burning and (2) the external capture of vapors outside of the vaporizing unit.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A vaporizer system for vaporizing a plant substance, comprising:
a heating system configured to generate heat;
a temperature control system configured to monitor the level of heat generated by the heating system;
a control unit in communication with the heating system and the temperature control system, the control unit configured to provide an interface for a user to regulate the performance of the temperature control system and the heating system;
a cooking unit in communication with the heating system and the temperature control system and configured to provide a fully enclosed volume to capture the heat generated by the heating system;
a valve body in communication with the cooking unit being configured to regulate the release of vapors within the cooking unit; and
a bag member in communication with the valve body for the collection of released vapors.

2. The system of claim 1, wherein the bag member is releasably coupled to the valve body.

3. The system of claim 1, wherein the bag member is contoured in the shape of a muffin.

3. The system of claim 1, wherein the bag member is externally located in relation to the cooking unit.

4. The system of claim 1, wherein the bag member is transportable separately from the cooking unit.

5. The system of claim 1, wherein the bag member is configured to permit the cooling of the vapor outside of the cooking unit.

6. The system of claim 1, wherein the control unit includes a digital interface.

7. The system of claim 1, wherein the control unit includes a manual interface.

8. The system of claim 1, wherein the control unit regulates the duration of heating from the heating system and the temperature levels within the cooking unit.

9. The system of claim 1, wherein the temperature control system is in communication with the control unit so as to provide temperature data, the control unit being configured to adjust the level of heat in the cooking unit as a result of the temperature data received.

10. The system of claim 1, wherein the cooking unit includes a door hinged along a lower edge to permit the selective access to the enclosed volume.

11. A method of heating a plant substance and capturing vapor, comprising:
obtaining the plant substance;
inserting the plant substance into a cooking unit;
heating the plant substance in the cooking unit via a heating system;
selectively releasing gas vapors from the heated plant substance through a valve body; and
capturing the gas vapors in a bag member.

12. The method of claim 11, further comprising:
regulating the temperature of the cooking unit through a temperature control system.

13. The method of claim 11, wherein the bag member is configured to permit the cooling of the vapor outside of a vaporizer unit, the vaporizer unit including the cooking unit, the heating system and the valve body.

14. The method of claim 13, wherein vaporizer unit is shaped as an oven.

15. The method of claim 11, wherein control of the heating system and the temperature control system is handled through a control unit configured to provide a user interface.

16. The method of claim 11, further comprising:
detaching the bag member from the valve body for selective inhalation of the gas vapor.
